(19) United States
(12) Patent Application Publication
(10) Pub. No.: US 2021/0407652 A1
(43) Pub. Date: Dec. 30, 2021

(54) HEALTHY BEHAVIOR SUPPORT DEVICE, METHOD, AND NON-TRANSITORY STORAGE MEDIUM STORING PROGRAM

(71) Applicant: OMRON HEALTHCARE Co., Ltd., Kyoto (JP)

(72) Inventors: Hiroshi Ogawa, Kyoto (JP); Miyuki Kimura, Kyoto (JP); Hironori Sato, Kyoto (JP); Atsunori Iwamoto, Kyoto (JP); Takashi Kitamura, Kyoto (JP); Tamio Ueda, Kyoto (JP); Mizuki Furuta, Kyoto (JP)

(21) Appl. No.: 17/447,365

(22) Filed: Sep. 10, 2021

Related U.S. Application Data
(63) Continuation of application No. PCT/JP2020/005957, filed on Feb. 17, 2020.

(30) Foreign Application Priority Data
Mar. 15, 2019 (JP) 2019-048355

(51) Int. Cl. G16H 20/70; G16H 50/30

(52) U.S. Cl. CPC G16H 20/70; G16H 50/30

(57) ABSTRACT

To effectively support behavior that maintains and promotes the health of a user, and to improve communication between users. A healthy behavior support device includes a processor. The processor is configured to select a plurality of comparison target users satisfying a predetermined condition from a storage unit that stores schedule information of a plurality of users, notify each of the selected plurality of comparison target users of a comparison counterpart user, acquire an index value correlated to state of health of each of the selected plurality of comparison target users, and set the acquired index values to a state of being sharable between the plurality of comparison target users.

### CROSS-REFERENCE TO RELATED APPLICATIONS

This application is the U.S. national stage application filed pursuant to 35 U.S.C. 365(c) and 120 as a continuation of International Patent Application No. PCT/JP2020/005957, filed Feb. 17, 2020, which application claims priority to Japanese Patent Application No. 2019-048355, filed Mar. 15, 2019, which applications are incorporated herein by reference in their entireties.

### TECHNICAL FIELD

One aspect of the present invention relates to a healthy behavior support device that supports behavior that maintains and promotes the health of a user, for example, and also relates to a method therefor and a non-transitory storage medium storing a program.

### BACKGROUND ART

Various techniques have been proposed to support behavior that helps maintain and promote the health of users in their daily lives. For example, Patent Document 1 describes a technique that causes a server to compare a step count sent from a user terminal to a predetermined condition and, if the condition is satisfied, for example, provided that a target step count is exceeded, the server awards points to the user and displays an interactive message on the user terminal.

### CITATION LIST

### Patent Literature

Patent Document 1: JP 2018-85083 A

### SUMMARY OF INVENTION

### Technical Problem

The technique described in Patent Document 1 manages the health of the user in a one-on-one interactive manner between a server and each user. Therefore, even though the server is a virtual interaction counterpart, the user is essentially left alone to take action for health maintenance and promotion without interacting with others, and this makes it difficult for the user to stay motivated for a long time.

The present invention has been made in view of the above situation, and aims to provide a technique that effectively supports behavior that maintains and promotes the health of a user and improves communication between users.

### Solution to Problem

To solve the above problem, a first aspect of the present invention is a healthy behavior support device including a processor, or a healthy behavior support method executed by the healthy behavior support device, in which the processor is configured to select a plurality of comparison target users satisfying a predetermined condition from a storage unit that stores schedule information of a plurality of users, notify each of the selected plurality of comparison target users of a comparison counterpart user, acquire an index value correlated to a state of health of each of the selected plurality of comparison target users, and set the acquired index values to a state of being sharable between the plurality of comparison target users.

According to the first aspect of the present invention, the plurality of comparison target users is selected from among the plurality of users according to their schedule information, and the index values correlated to the state of health of the individual comparison target users can be compared between the plurality of comparison target users. This allows the users to interact with acquaintances who have the same schedule by stimulating each other to maintain and promote their state of health. Compared to a case where users take actions for health maintenance and promotion by themselves, the users can continue healthy behaviors for a longer term and expect a higher health maintenance and promotion effect. Improved communication between users through such mutual healthy behavior can also be expected.

A second aspect of the present invention is to select, from the storage unit storing the schedule information, a plurality of the users scheduled to perform a common action during a predetermined time segment as the comparison target users in selection processing of selecting the plurality of comparison target users.

According to the second aspect of the present invention, the plurality of users scheduled to perform the common action during the predetermined time segment are selected as the comparison target users according to the schedule information. This allows the users scheduled to participate in, for example, the same meeting to be selected as the comparison target users, so that the users can communicate with each other when, for example, they meet for the meeting and talk about their healthy behaviors.

A third aspect of the present invention is to select, from the storage unit storing the schedule information, a plurality of users having performed no common action for at least a predetermined period of time as the comparison target users in the selection processing of selecting the plurality of comparison target users.

According to the third aspect of the present invention, the plurality of users who has not performed the common action for the predetermined period of time or longer is selected as the comparison target users, so that, as in a case where the users belong to a common community and can refer to each other's schedule but have few opportunities to perform activities together, the users can enhance communication with each other by talking about their healthy behaviors.

A fourth aspect of the present invention is that the processor further performs processing of comparing the acquired index values or values representing a degree of health calculated based on the index values, and ranking the plurality of comparison target users based on a result of the comparison according to a predetermined rule.

According to the fourth aspect of the present invention, the acquired index values or the values representing the degree of health of the plurality of comparison target users are compared, and the plurality of comparison target users is ranked based on the result of the comparison. For example, when there are many comparison target users, each user can easily check the position of his/her own healthy behavior efforts among all the comparison target users according to the ranking information.

A fifth aspect of the present invention is to transmit, at predetermined timing, at least one of comparison data of the acquired index values of the plurality of comparison target users, comparison data of values representing the degree of health calculated based on the index values, and data representing a list ranking the plurality of comparison target users, to terminals used by the plurality of comparison target users.

According to the fifth aspect of the present invention, the healthy behavior support device automatically notifies each terminal of the comparison target users of the comparison data between the comparison target users, for example, during or at the end of the execution of the healthy behavior. Thus, each of the comparison target users can easily acquire and confirm the comparison data against other comparison target users without any particular operation.

A sixth aspect of the present invention is to save, in a storage medium, at least one of comparison data of the acquired index values of the plurality of comparison target users, comparison data of values representing the degree of health calculated based on the index values, and data representing a list ranking the plurality of comparison target users, and transmit the saved comparison data to a requesting user terminal in response to a viewing request from the terminals used by the comparison target users.

According to the sixth aspect of the present invention, the comparison target users can perform a viewing operation, for example, on their terminals to view and confirm the comparison data of the comparison target users at their desired timing.

A seventh aspect of the present invention is to acquire at least one of a step count, a traveled distance, an amount of activity, calorie intake, and biometric information in the acquisition processing to acquire the index values.

According to the seventh aspect of the present invention, the values representing the behavioral state of users such as the step count, the traveled distance, the amount of activity, and the calorie intake, and the values representing biometric information that reflects their healthy behavior, such as blood pressure values and blood glucose values, can be used as the index values correlated to the state of health of the comparison target users.

### Advantageous Effects of Invention

According to the aspects of the present invention, a technique of effectively supporting behavior that maintains and promotes the health of a user and improves communication between users can be provided.

### BRIEF DESCRIPTION OF DRAWINGS

FIG. 1 is a block diagram illustrating an application example of a healthy behavior support device according to an embodiment of the present invention.

FIG. 2 illustrates a configuration example of a system that includes the healthy behavior support device according to the embodiment of the present invention.

FIG. 3 is a block diagram illustrating an example of a hardware configuration of a user terminal used in the system illustrated in FIG. 2.

FIG. 4 is a block diagram illustrating an example of a software configuration of the user terminal used in the system illustrated in FIG. 2.

FIG. 5 is a block diagram illustrating an example of a hardware configuration of a healthy behavior support server used in the system illustrated in FIG. 2.

FIG. 6 is a block diagram illustrating an example of a software configuration of the healthy behavior support server used in the system illustrated in FIG. 2.

FIG. 7 is a flowchart illustrating a first half of the procedure and details of processing executed by the user terminal illustrated in FIG. 4 and the healthy behavior support server illustrated in FIG. 6.

FIG. 8 is a flowchart illustrating a second half of the procedure and details of processing executed by the user terminal illustrated in FIG. 4 and the healthy behavior support server illustrated in FIG. 6.

FIG. 9 is a display example of step count comparison data generated by the processing illustrated in FIG. 8.

FIG. 10 is a display example of a step count ranking generated by the processing illustrated in FIG. 8.

### DESCRIPTION OF EMBODIMENTS

An embodiment according to one aspect of the present invention will be described below with reference to the accompanying drawings. Note that the embodiment described below is merely an illustration of the invention in all respects.

### Application Example

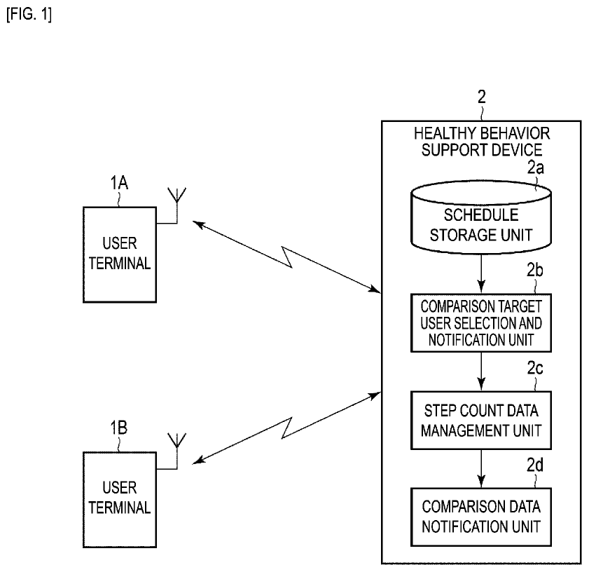

First, an application example of a healthy behavior support device according to an embodiment of the present invention is described. FIG. 1 is an explanatory view illustrating an application example of a healthy behavior support device according to an embodiment of the present invention including user terminals 1A and 1B and a healthy behavior support device 2.

The user terminals 1A and 1B are each constituted by a portable information terminal such as a smart phone or a wearable terminal, and have a counting function to count the number of steps, a browser function to access the Web, and a mailer function to transmit and receive emails.

The healthy behavior support device 2 is constituted by, for example, a server computer, and includes a schedule storage unit 2a, a comparison target user selection and notification unit 2b, a step count data management unit 2c, and a comparison data notification unit 2d.

The schedule storage unit 2a stores, for example, schedule information of a plurality of users who belongs to the same community such as a workplace, a school, a neighborhood association, or a club. The users whose schedule information is to be stored do not necessarily need to belong to the same community, but the users need to have a relationship in which the users can refer to each other's schedule information.

According to the schedule information stored in the schedule storage unit 2a, the comparison target user selection and notification unit 2b selects a plurality of users, for example, scheduled to participate in the same meeting on the same date and time from among a plurality of users, and saves information of the plurality of selected users in a memory of the device 2 as comparison target users who participate in a walking exercise (walking challenge) event, while also transmitting the information to the corresponding user terminals 1A and 1B.

Two users are selected as the comparison target users in this example, but three or more users may be selected. Alternatively, four or more selected users, for example, may be divided into a plurality of user groups, and these user groups may be used as the comparison target users.

The step count data management unit 2c receives the step count value measurement data (step count data) of the users, which is transmitted from the user terminals 1A and 1B of the plurality of comparison target users during implementation of the walking exercise event, and saves the step count data in the memory in the device 2. The comparison data notification unit 2d generates step count comparison data used for listing and displaying the step count value data of the comparison target users in a comparable state, and transmits the step count comparison data to the user terminals 1A and 1B of the comparison target users. The transmission timing can be during the event implementation period, after the event implementation period, or both. In addition, the comparison data notification unit 2d performs processing of notifying the user terminals 1A and 1B of the selection result of the comparison target users at or before the start of the event implementation period.

In the configuration described above, when the walking exercise event starts, the healthy behavior support device 2 first selects, for example, two users who are scheduled to participate in the same meeting on the same date and time according to the schedule information and notifies the two users of being selected as the comparison target users. During the implementation period of the event from the start time to a predetermined end time of the event, the healthy behavior support device 2 performs processing of acquiring, from the user terminals 1A and 1B, the step count data of the comparison target users measured by the user terminals 1A and 1B, generating the step count comparison data for listing and displaying the step count data in a comparable state, and then transmitting the step count comparison data to the user terminals 1A and 1B.

Thus, each comparison target user is notified of the data in the comparable state, whereby each comparison target user can compare his/her step count value with the step count values of the comparison counterpart users. Each comparison target user can perform walking exercise while comparing his/her own step count value to the step count values of the comparison counterpart users during or after the event implementation period. This allows the users to interact with acquaintances who belong to the same community and have the same schedule that the day by stimulating each other to maintain and promote their state of health. Compared to a case where users take actions for health maintenance and promotion by themselves, the users can continue the healthy behavior for a longer term and expect a higher health maintenance and promotion effect. Improved communication between users through mutual healthy behavior can also be expected.

### Embodiment

### Configuration Example

### (1) System

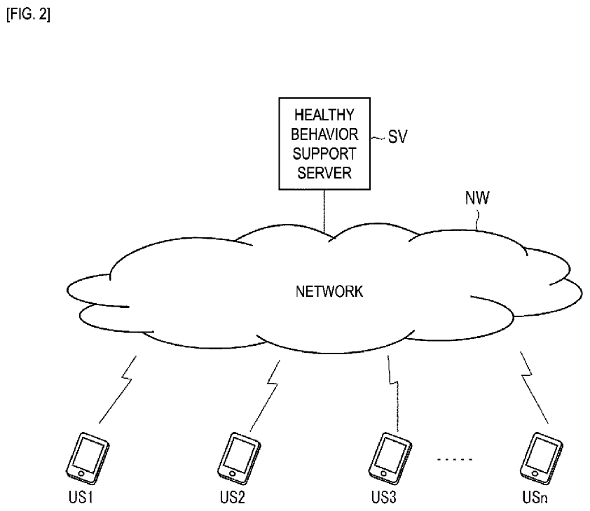

FIG. 2 illustrates an overall configuration of a system provided with a healthy behavior support device according to an embodiment of the present invention, including a server (healthy behavior support server) SV as a healthy behavior support device, and terminals (user terminals) US1 to USn used by a plurality of users who are candidates for the comparison target users. In this example, a network NW is a wireless local area network (LAN) set up in a building or an office. Alternatively, the network NW may be constituted by the Internet and various access networks used for accessing the Internet.

### (2) User Terminal US1 to USn

### (2-1) Hardware Configuration

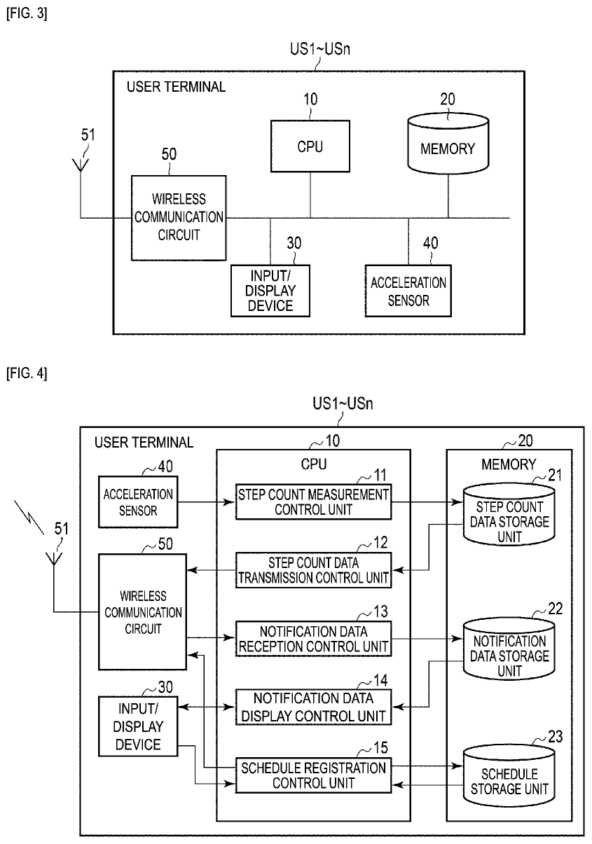

FIG. 3 is a block diagram illustrating an example of a hardware configuration of the user terminals US1 to USn.

The user terminals US1 to USn each include, for example, a hardware processor 10 such as a central processing unit (CPU) (hereinafter the hardware processor may also be referred to as the CPU), and the hardware processor 10 is connected to a memory 20, an input/display device 30, an acceleration sensor 40, and a wireless communication circuit 50 via a bus.

The memory 20 uses a non-volatile memory, such as a solid state drive (SSD), as a storage medium that can be written and read as needed and includes a program storage area and a data storage area. The program storage area stores a middleware program functioning as an operating system (OS) and various application programs. In the data storage area, various kinds of data including step count data and notification data sent from the healthy behavior support server SV are saved.

The input/display device 30 is constituted by, for example, a tablet-type device with a capacitive or pressure-sensitive input sheet layered on a display screen made of liquid crystal or organic EL, and is used to input operational information by the user and display the display data.

The acceleration sensor 40 is constituted by, for example, a three-axis acceleration sensor and detects acceleration in the three axial directions of the user terminals US1 to USn. Detected acceleration values in the three-axis directions are used by the CPU 10 to measure the step count.

The wireless communication circuit 50 is used to execute wireless communication with access points of the network NW in accordance with, for example, a wireless communication method defined in the wireless LAN. Typically, the CPU 10 controls protocols and the like of the wireless communication, but the wireless communication circuit 50 may also control these. Note that an antenna 51 is also illustrated.

### (2-2) Software Configuration

FIG. 4 is a block diagram illustrating a software configuration of the user terminals US1 to USn in relation to the hardware configuration illustrated in FIG. 3.

The memory 20 includes a step count data storage unit 21, a notification data storage unit 22, and a schedule storage unit 23 in the data storage area. The step count data storage unit 21 is used to store a measured step count value in relation to its detection time. The notification data storage unit 22 is used to save various kinds of notification data transmitted from the healthy behavior support server SV. The schedule storage unit 23 is used to store a personal schedule of each user registered by that user.

The CPU 10 functions as a processing functional unit according to the embodiment, and includes a step count measurement control unit 11, a step count data transmission control unit 12, a notification data reception control unit 13, a notification data display control unit 14, and a schedule registration control unit 15. All of these control units 11 to 15 are implemented by causing the CPU 10 to execute application programs stored in the program storage area in the memory 20.

The step count measurement control unit 11 captures a three-axis acceleration signal output from the acceleration sensor 40, extracts a feature representing the walking motion of the user from the three-axis acceleration signal, and calculates the step count value of the user by time segment based on the extraction result of the feature. The step count measurement control unit 11 then causes the step count data storage unit 21 to store the step count value calculated for each time segment.

The step count data transmission control unit 12 reads, from the step count data storage unit 21, step count values for predetermined time intervals during the implementation period of the walking exercise event, at those time intervals. Then, the step count data transmission control unit 12 generates the step count data including the step count value for each time interval, information representing the measurement time, and user identification information (user ID), and controls the step count data to be transmitted to the healthy behavior support server SV. The wireless communication circuit 50 converts the step count data into a wireless signal and transmits this signal from the antenna 51 under the control of the step count data transmission control unit 12.

The notification data reception control unit 13 performs processing of receiving notification data transmitted from the healthy behavior support server SV via the wireless communication circuit 50, and saving the notification data in the notification data storage unit 22. The notification data display control unit 14 performs processing of reading the notification data from the notification data storage unit 22, and displaying the notification data on the input/display device 30.

The schedule registration control unit 15 follows an application program (scheduler) used for registering a schedule to store the schedule information of the user, which is entered by the user from the input/display device 30, in the schedule storage unit 23, while transmitting the schedule information from the wireless communication circuit 50 to the healthy behavior support server SV.

### (3) Healthy Behavior Support Server SV

### (3-1) Hardware Configuration

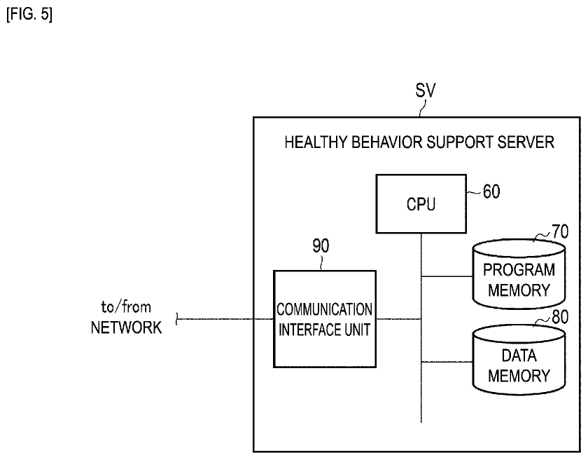

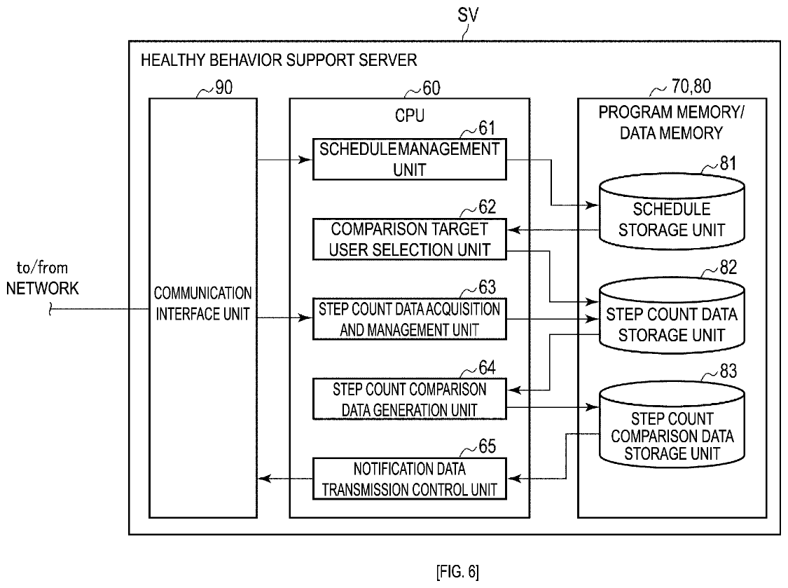

FIG. 5 is a block diagram illustrating an example of a hardware configuration of the healthy behavior support server SV.

The healthy behavior support server SV includes a hardware processor, such as a CPU (hereinafter the hardware processor may also be referred to as the CPU) 60, and the hardware processor 60 is connected to a program memory 70, a data memory 80, and a communication interface unit 90 via a bus.

The communication interface unit 90 transmits data between the user terminals US1 to USn in accordance with, for example, a communication protocol defined in the network NW.

The program memory 70 is a storage medium that uses, for example, both a non-volatile memory that can be written and read as needed, such as an HDD or an SSD, and a non-volatile memory such as a ROM, and stores programs necessary for executing various kinds of control processing according to the embodiment.

The data memory 80 includes a storage medium constituted by a combination of a non-volatile memory that can be written and read as needed, such as an HDD or an SSD, and a volatile memory such as a random access memory (RAM). The data memory 80 is used to save various kinds of data necessary to implement the embodiment.

### (3-2) Software Configuration

FIG. 6 is a block diagram illustrating a software configuration of the healthy behavior support server SV in relation to the hardware configuration illustrated in FIG. 5.

The data memory 80 includes a schedule storage unit 81, a step count data storage unit 82, and a step count comparison data storage unit 83. The schedule storage unit 81 is used to store the schedule information sent from the individual user terminals US1 to USn for each user.

The step count data storage unit 82 is used to store the step count data of the comparison target users sent from the user terminals US1 to USn in relation to identification information (user ID) of the comparison target users selected by the CPU 60. The step count comparison data storage unit 83 is used to store step count comparison data representing comparison results of the step counts of the comparison target users, and step count ranking data ranking the comparison target users according to their step counts.

The CPU 60 is a processing functional unit according to the embodiment and includes a schedule management unit 61, a comparison target user selection unit 62, a step count data acquisition and management unit 63, a step count comparison data generation unit 64, and a notification data transmission control unit 65. All of these control processing functional units 61 to 65 are implemented by causing the CPU 60 to execute programs stored in the program memory 70.

The schedule management unit 61 performs processing of transmitting template data for the input of the schedule in response to a request from the user terminals US1 to USn, receiving the schedule information input by the user according to the template data from the user terminals US1 to USn, and storing the schedule information in relation to the user IDs in the schedule storage unit 81.

The comparison target user selection unit 62 selects, as the comparison target users, a plurality of users scheduled to participate in, for example, the same meeting on the same date and time according to the schedule information of each user stored in the schedule storage unit 81 at or prior to the start of the walking exercise event for each unit time segment, such as a day, a week, or a month, in which the event is implemented. The number of users to be selected may be two users or three or more users.

The comparison target user selection unit 62 also performs processing of storing information representing the selection result of the comparison target users, for example, information representing the details of a pair of users when two comparison target users are selected, or information representing the details of a group of users when three or more comparison target users are selected, in the step count data storage unit 82.

Furthermore, the comparison target user selection unit 62 performs processing of reading, from the schedule storage unit 81, attribute information including names of the users and names of departments to which individual users belong for the pair or group of users, and transmitting notification data including the attribute information to the corresponding user terminals US1 to USn from the communication interface unit 90.

The step count data acquisition and management unit 63 receives the step count data of the users periodically transmitted, for example, at predetermined time intervals from the user terminals US1 to USn of the individual comparison target users who constitute the pair or group during the implementation period of the walking exercise event. In this example, the step count data includes the step count values, information representing the measurement period of the step count values, and the user IDs. Then, the step count data acquisition and management unit 63 associates the received step count data with the user IDs of the comparison target users stored in the step count data storage unit 82 and stores the step count data in the step count data storage unit 82.

The step count comparison data generation unit 64 performs processing of reading the step count data of the individual users constituting the pair or group among the comparison target users from the step count data storage unit 82 for each pair or group, for example, at a predetermined time during and at the end of the implementation period of the walking exercise event, generating the step count comparison data listing the step count data of the corresponding users in a comparable manner, and storing the step count comparison data in the step count comparison data storage unit 83.

In addition, the step count comparison data generation unit 64 performs processing of generating ranking data of the step counts, for example, by department to which the users belong by ranking the step count values of the comparison target users stored in the step count data storage unit 82 in descending order relative to the step count data, and storing the ranking data in the step count comparison data storage unit 83.

The notification data transmission control unit 65 performs processing of reading the step count comparison data from the step count comparison data storage unit 83 for each pair or group of the comparison target users, and transmitting the step count comparison data to the corresponding user terminals at a predetermined notification timing. The notification timing can be during the event implementation period, after the implementation period, or both.

When a viewing request is sent from the user terminal US1 to USn, the notification data transmission control unit 65 also performs processing of reading, from the step count comparison data storage unit 83, the step count comparison data corresponding to the comparison target users specified by the request, and transmitting the step count comparison data to the requesting user terminal.

### Operation Example

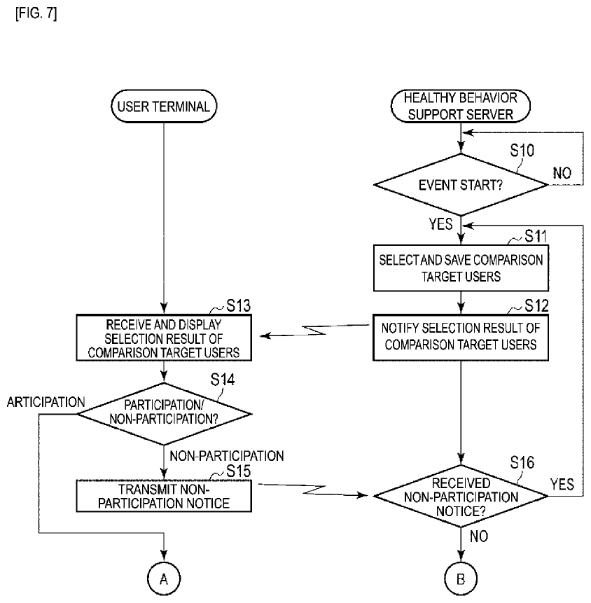

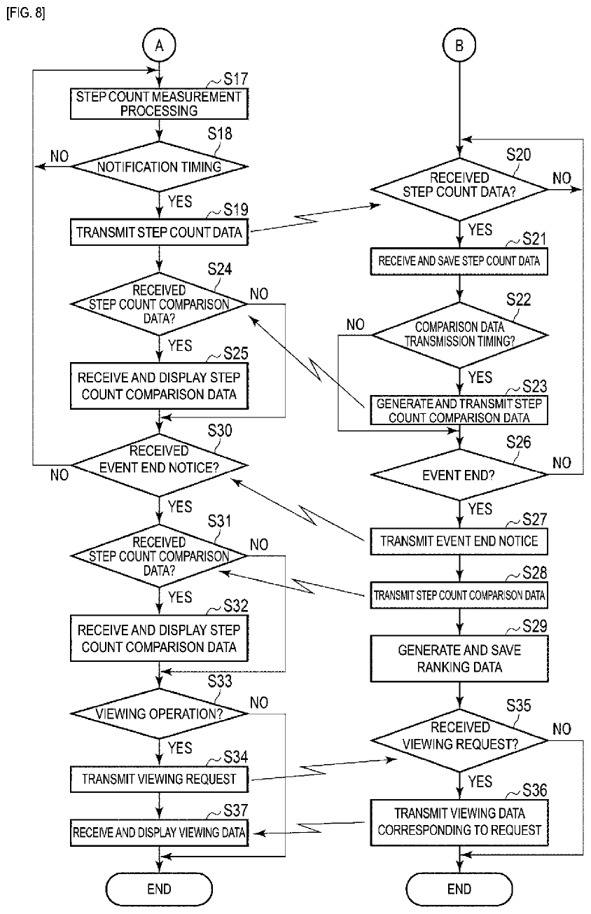

Next, the operation of the user terminals US1 to USn and the healthy behavior support server SV configured as described above is described. FIGS. 7 and 8 are flowcharts illustrating an operation example.

In this example, all employees working in the same workplace are regarded as the target users who participate in the walking exercise event, and the walking exercise event is implemented for a day as unit time segment.

### (1) Registration and Management of Schedules

Before the start of the walking exercise event, the user terminals US1 through USn and the healthy behavior support server SV are subjected to registration and management processing of the schedules of the users. Specifically, when the user launches the application program for schedule registration on his/her user terminal US1 to USn, personal schedule information input by the user to the input/display device 30 is stored in the schedule storage unit 23 under the control of the schedule registration control unit 15. When the registration processing of the personal schedule information of, for example, a day is completed, the registered personal schedule information is read from the schedule storage unit 23 and transmitted along with the user ID from the wireless communication circuit 50 to the healthy behavior support server SV under the control of the schedule registration control unit 15.

On the other hand, when the healthy behavior support server SV receives the personal schedule information transmitted from the user terminals US1 to USn at the communication interface unit 90, the personal schedule information is stored in the schedule storage unit 81 in relation to the users ID under the control of the schedule management unit 61.

Subsequently, the personal schedule information of individual users is added to or updated in the schedule storage units 23 and 81 as needed according to the registration operation by the users.

### (2) Selection and Notification of Comparison Target Users

When the predetermined event start time arrives, the healthy behavior support server SV shifts the process from step S10 to step S11 under the control of the comparison target user selection unit 62, and selects the comparison target users according to the schedule information of individual users stored in the schedule storage unit 81.

For example, the personal schedule information of the users stored in the schedule storage unit 81 is compared to select a plurality of users scheduled to attend the same meeting held on the day of the event as the comparison target users. The number of users to be selected may be two users or three or more users. When two users are selected, the comparison target user selection unit 62 configures these users as a pair of comparison target users. When three users are selected, the comparison target user selection unit 62 configures these users as a group of comparison target users. Four or more users, for example, may also be selected and divided into a plurality of groups.

The comparison target user selection unit 62 stores information representing the configuration of the pair or group of the comparison target users in the step count data storage unit 82. In addition, the comparison target user selection unit 62 reads attribute information including the name, the department, and the like of each user constituting the pair or group from the schedule storage unit 81 in step S12. Then, comparison target notification data including the attribute information is transmitted from the communication interface unit 90 to the corresponding user terminals US1 to USn. For example, an email or a short message is used as the transmission method of the comparison target notification data.

On the other hand, the user terminals US1 to USn, when receiving the comparison target notification data transmitted from the healthy behavior support server SV at the wireless communication circuit 50, saves the received comparison target notification data in the notification data storage unit 22 in step S13 under the control of the notification data reception control unit 13, and displays the comparison target notification data on the input/display device 30 under the control of the notification data display control unit 14. As a result, the users can confirm the comparison target users to be comparison counterpart users in the walking exercise event on that day.

In response to the displayed comparison target users, assume that the user enters “will not participate in the walking exercise event” to his/her user terminal, because the user intends to take a day off today. In this case, the user terminal transmits a notice of “will not participate in the walking exercise event” to the healthy behavior support server SV under the control of the schedule registration control unit 15 in step S15.

When the healthy behavior support server SV detects the receipt of the non-participation notice transmitted from the user terminal in step S16, the process returns to step S11. Then, under the control of the comparison target user selection unit 62, the comparison target users are re-selected in steps S11 and S12 by excluding the non-participation user, and the result is transmitted to the corresponding user terminals.

### (3) Operation During Event Implementation Period

Assume that the user enters participation to his/her user terminal US1 to USn in response to the displayed comparison target users. Then, the user terminal US1 to USn executes step count measurement processing as described below under the control of the step count measurement control unit 11 in step S17. For example, the user terminal US1 to USn captures the three-axis acceleration signal output from the acceleration sensor 40 and extracts a feature representing the walking motion of the user from the three-axis acceleration signal. Based on the extraction result of the feature, the user terminal US1 to USn calculates the step count value for each time segment, for example, an hourly step count value of the user. The step count measurement control unit 11 then stores the calculated step count values by time segment in the step count data storage unit 21 in association with the information representing the time segment. The step count measurement processing continues until the predetermined notification timing, for example, every hour on the hour, is detected in step S18.

At the notification timing, the user terminal US1 to USn reads the step count data for the past one hour from the step count data storage unit 21 and transmits the step count data together with the user ID and time stamp information indicating the measurement time from the wireless communication circuit 50 to the healthy behavior support server SV under the control of the step count data transmission control unit 12 in step S19.

On the other hand, the healthy behavior support server SV monitors receipt of the step count data in step S20. In this state, when the step count data transmitted from the user terminal US1 to USn is received at the communication interface unit 90, the healthy behavior support server SV receives the step count data and stores this data in the step count data storage unit 82 under the control of the step count data acquisition and management unit 63 in step S21. Subsequently, the healthy behavior support server SV similarly receives the step count data every time the step count data is transmitted from the user terminals US1 to USn, and stores the step count data in the step count data storage unit 82. The step count data of all users participating in the event is sequentially stored every hour in the step count data storage unit 82 of the healthy behavior support server SV.

During the event implementation period, the healthy behavior support server SV also monitors the transmission timing of the comparison data for each pair or group of the comparison target users in step S22. In this state, when the predetermined transmission timing, for example, time set every three hours, is reached, the healthy behavior support server SV reads the step count data of the individual users constituting the pair or group of the comparison target users from the step count data storage unit 82 for each pair or group under the control of the step count comparison data generation unit 64 in step S23. Then, the healthy behavior support server SV generates the step count comparison data listing and displaying the step count data in a comparable manner and transmits the data to the corresponding user terminals.

On the other hand, when the user terminal US1 to USn detects the receipt of the step count comparison data transmitted from the healthy behavior support server SV in step S24 at the wireless communication circuit 50, the user terminal US1 to USn receives the step count comparison data and saves this data in the notification data storage unit 22 under the control of the notification data reception control unit 13 in step 25, reads the step count comparison data from the notification data storage unit 22 and displays the data on the input/display device 30 under the control of the notification data display control unit 14.

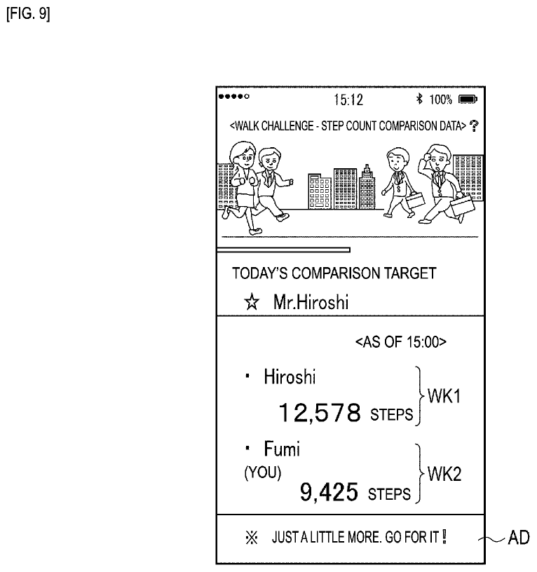

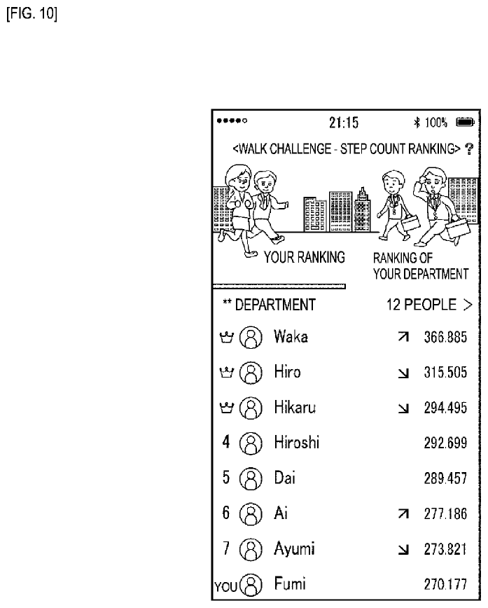

FIG. 9 shows an example of a display result of the step count comparison data of two users constituting a pair. As shown in the figure, the step count comparison data is marked with the names and the step count values WK1 and WK2 of the two users constituting the pair. A message of support AD for the users may also be displayed. As a result, the user can compare the progress of his/her own step count value and that of the comparison counterpart user during the event implementation period and easily check the difference or the like between the two step counts.

(4) Operation after End of Event Implementation Period

In step S26, the healthy behavior support server SV monitors whether the event end time has been reached. If the event end time has not yet been reached, i.e., if the event is still in progress, the process returns to step S20, and the acquisition of the step count data and the generation and notification of the step count comparison data are repeated in steps S20 to S23 as described above.

When the event end time is detected in step S26, the healthy behavior support server SV transmits an event end notice to the user terminals US1 to USn in step S27. At the same time, the healthy behavior support server SV transmits the step count comparison data as of the end of the event generated by the step count comparison data generation unit 64 to the user terminals US1 to USn in step S28.

In addition, the healthy behavior support server SV generates the ranking data of the step counts by ranking the step count values of the comparison target users stored in the step count data storage unit 82 in descending order relative to the step count data, for example, by department to which the users belong under the control of the step count comparison data generation unit 64 in step S29. Then, the ranking data is stored in the step count comparison data storage unit 83.

The user terminals US1 to USn are monitoring whether the event end notice has been received in step S30. Until the event end notice is received, the process returns to step S17, and the measurement and notification of the step count data and the receipt and display of the step count comparison data in steps S17 to S25 are repeated.

Once the event end notice is received, the user terminal US1 to USn receives the step count comparison data as of the end of the event transmitted from the healthy behavior support server SV, stores the step count comparison data in the notification data storage unit 22 in step S31 under the control of the notification data reception control unit 13, and displays the step count comparison data on the input/display device 30 under the control of the notification data display control unit 14 in step S32. As a result, immediately after the end of the event, the user can check his/her own step count value and the step count value of the comparison counterpart as of the end of the event.

### (5) Viewing Step Count Comparison Data

Assume that, after the event ends, the user performs an operation to request from his/her user terminal US1 to USn to view the step count comparison data or ranking data by specifying the date and time. In this case, upon detection of the viewing request operation in step S33, the user terminal US1 to USn generates the viewing request and transmits this request to the healthy behavior support server SV in step S34. The viewing request includes a desired date for viewing, the type of data to be viewed, and the user ID.

The healthy behavior support server SV receives the viewing request in step S35 and reads the corresponding step count comparison data or ranking data from the step count comparison data storage unit 83 in step S36 under the control of the notification data transmission control unit 65 based on the desired date for viewing, the type of data to be viewed, and the user ID included in the viewing request. Then, the healthy behavior support server SV transmits the read step count comparison data or ranking data to the requesting user terminal US1 to USn.

The user terminal US1 to USn receives the step count comparison data or ranking data transmitted from the healthy behavior support server SV and saves this data in the notification data storage unit 32 in step S37 under the control of the notification data reception control unit 13, and displays the step count comparison data or ranking data on the input/display device 30 under the control of the notification data display control unit 14.

FIG. 10 is a display example of one set of the ranking data on the user terminal US1 to USn. As shown in the figure, the ranking data consists of the names and step count values of the plurality of users who belong to the same department, sorted in descending order of the step count values, so that the user can easily check how high his/her own step count value is in the department to which he/she belongs according to the ranking. In the ranking data, arrows indicating an increase or a decrease of the step count value of each user from the previous day are also added, as shown in FIG. 10.

### Actions and Effects

According to the embodiment as described above, in providing the service of the walking exercise event to the plurality of users who belong to the same workplace, the healthy behavior support server SV selects, prior to the start of the event, a plurality of users scheduled to participate in the same meeting on the same day as the comparison target users according to the schedule information of the plurality of users, notifies the users of the selection result, then collects the measured step count values of the comparison target users from the user terminals US1 to USn to generate the step count comparison data, and notifies each user of the comparison data.

Therefore, the user can perform walking exercise while comparing his/her own step count value with that of the other users who are comparison counterpart users, and can interact with acquaintances who belong to the same workplace and have the same schedule for the day by stimulating each other to maintain and improve their states of health. As a result, compared to a case where the users perform the walking exercise by themselves, the users can continue the walking exercise for a longer period and expect a higher health maintenance and promotion effect. Improvement of communication between users through the walking exercise can also be expected.

Further, according to the embodiment, the healthy behavior support server SV automatically notifies each user of the step count comparison data at an appropriate time during the event implementation period and at the end of the event, so that users can maintain their interest in walking exercise throughout the day.

Further, according to the embodiment, the data representing the ranking of the step counts of the day is generated and saved for each department after the event, and the ranking data is transmitted to the user terminals US1 to USn in response to the viewing request from the user terminals US1 to USn. This allows the users to manage the results of their own walking exercise efforts by comparing the results of other users in the same department.

### Modification Examples

(1) The embodiment deals with a case where the transmission timing of the step count data from the user terminals US1 to USn to the healthy behavior support server SV is commonly set in the user terminals US1 to USn. Alternatively, the same transmission timing may be set for users constituting a pair or group of the comparison target users, while different transmission timings may be set between different pairs or groups of the comparison target users. This unifies the step count values of the individual users constituting the pair or group with the values measured in the same measurement period to maintain ease of comparison, while transmitting the step count data at different transmission timings between different pairs or groups of the comparison target users, thus reducing the communication traffic involved in the network NW and lowering the processing load correlated to the step count data acquisition management processing of the healthy behavior support server SV.
(2) The embodiment deals with a case where the plurality of users scheduled to participate in the same meeting on the same day is selected as the pair or group of the comparison target users according to the schedule information. Alternatively, the healthy behavior support server SV may select a plurality of users who are presumed not to have met each other for at least a predetermined period of time as the pair or group of comparison target users according to the schedule information. This allows the information of the step count values of the walking exercise event to be shared by the users, thus increasing opportunities for communication between users who have few opportunities to contact each other due to the difference in their type of work even within the same organization, or users who have become estranged from each other.
(3) The embodiment deals with a case where the step count values between individual users can be compared. Alternatively, a total number of step count values of the individual groups may be compared among the plurality of groups each consisting of the plurality of users. This allows implementation of a so-called team competition for the walking exercise in which, for example, the departments compete against each other for the total step count in the walking exercise event to also promote participation in the walking exercise, so that further health maintenance and promotion of the users can be expected.
(4) The embodiment deals with a case where the healthy behavior support server SV executes the selection processing of the comparison target users according to the schedule information and the generation and notification processing of the step count comparison data. Alternatively, for example, one of the user terminals may be configured to have the above processing functions.
(5) The embodiment deals with a case where the schedule information is stored in the healthy behavior support server SV. Alternatively, the schedule information may be stored in an external device other than the healthy behavior support server SV, such as a cloud or web database, or a server or the like dedicated to schedule management. When using schedule information stored in such an external device, the schedule information may be referenced at the timing of selecting the comparison target users. Accordingly, the users who may not be included in an established community and do not known each other but may possibly get to know each other in the future can be selected as the comparison targets, so that improvement of communication among a wider range of users can be expected. In this case, the healthy behavior support server SV also stores information that identifies each user such as the name of the user in association with the information for accessing the schedule information of the users. To use the schedule information stored in the external device, the healthy behavior support server SV downloads the schedule information from the external device and stores the schedule information temporarily in the memory of the server SV for use.
(6) While embodiments of the present invention have been described in detail above, the foregoing description is merely an illustration of the present invention in all respects. Of course, various modifications and variations can be made without departing from the scope of the present invention. Thus, specific configurations in accordance with an embodiment may be adopted as appropriate at the time of carrying out the present invention.

In short, the invention is not limited to the embodiments described above and can be embodied by modifying the components in an implementation stage in a range without departing from the gist thereof. Additionally, various inventions can be formed by appropriately combining a plurality of components disclosed in the embodiments described above. For example, some components may be omitted from all the components described in the embodiments. Further, the components of the different embodiments may be combined appropriately.

### Supplementary Notes

A portion or all of the embodiments can be described as described in the following supplementary notes in addition to the scope of the claims, but is not limited to the supplementary notes.

### Supplementary Note 1

A healthy behavior support device includes:

a selection unit configured to select a plurality of comparison target users satisfying a predetermined condition from a storage unit that stores schedule information of a plurality of users,
a notification unit configured to notify each of the selected plurality of comparison target users of a comparison counterpart user,
an acquisition unit configured to acquire an index value correlated to a state of health of each of the selected plurality of comparison target users, and
a setting unit configured to set the acquired index value to a sharable state between the plurality of comparison target users.

### REFERENCE SIGNS LIST

1A, 1B, US1 to USn . . . User terminal
2, SV . . . Healthy behavior support device, Healthy behavior support server
2a, 81 . . . Schedule storage unit, Schedule storage unit
2b . . . Comparison target user selection and notification unit
2c . . . Step count data management unit
2d . . . Comparison data notification unit
10 . . . CPU of user terminal
11 . . . Step count measurement control unit
12 . . . Step count data transmission control unit
13 . . . Notification data reception control unit
14 . . . Notification data display control unit
15 . . . Schedule registration control unit
20, 70, 80 . . . Memory, Program memory, Data memory
21 . . . Step count data storage unit
22 . . . Notification data storage unit
23 . . . Schedule storage unit
30 . . . Input/display device
40 . . . Acceleration sensor
50 . . . Wireless communication circuit
51 . . . Antenna
60 . . . CPU of healthy behavior support server
61 . . . Schedule management unit
62 . . . Comparison target user selection unit
63 . . . Step count data acquisition and management unit
64 . . . Step count comparison data generation unit
65 . . . Notification data transmission control unit
81 . . . Schedule storage unit
82 . . . Step count data storage unit
83 . . . Step count comparison data storage unit
90 . . . Communication interface unit

1. A healthy behavior support device comprising a processor, the processor configured to execute:
selection processing of selecting a plurality of comparison target users satisfying a predetermined condition from schedule information of a plurality of users;
notifying processing of notifying each of the selected plurality of comparison target users of a comparison counterpart user;
acquisition processing of acquiring an index value correlated to a state of health of each of the selected plurality of comparison target users; and
setting processing of setting the acquired index value to a state of being sharable between the plurality of comparison target users, wherein
in the selection processing of selecting the plurality of comparison target users, a plurality of users scheduled to perform a common action during a predetermined time segment is selected as the comparison target users from the schedule information.

2. A healthy behavior support device, comprising a processor, the processor configured to execute:
selection processing of selecting a plurality of comparison target users satisfying a predetermined condition from schedule information of a plurality of users;
notifying processing of notifying each of the selected plurality of comparison target users of a comparison counterpart user;
acquisition processing of acquiring an index value correlated to a state of health of each of the selected plurality of comparison target users; and
setting processing of setting the acquired index value to a state of being sharable between the plurality of comparison target users, wherein
in the selection processing of selecting the plurality of comparison target users, a plurality of users having performed no common action for at least a predetermined period of time is selected as the comparison target users from the schedule information.

3. The healthy behavior support device according to claim 1, wherein
the processor further executes comparison processing of comparing the acquired index values or values representing a degree of health calculated based on the index value, and ranking the plurality of comparison target users based on a result of the comparison according to a predetermined rule.

4. The healthy behavior support device according to claim 3, wherein
the setting processing involves transmitting, at a predetermined timing, at least one of comparison data of the acquired index values of the plurality of comparison target users, comparison data of values representing the degree of health calculated based on the index values, and data representing a list of ranking of the plurality of comparison target users, to terminals used by the plurality of comparison target users.

5. The healthy behavior support device according to claim 3, wherein
the setting processing involves saving, in a storage medium, at least one of comparison data of the acquired index values of the plurality of comparison target users, comparison data of values representing the degree of health calculated based on the index values, and data representing a list of ranking of the plurality of comparison target users, and transmitting the saved comparison data to a requesting user terminal in response to a viewing request from terminals used by the plurality of comparison target users.

6. The healthy behavior support device according to claim 1, wherein
the acquisition processing of acquiring the index value involves acquiring at least one of a step count, a traveled distance, an amount of activity, calorie intake, and biometric information, as the index value.

7. A healthy behavior support method executed by a device including at least one processor, comprising:
selecting, by the processor, a plurality of comparison target users satisfying a predetermined condition from schedule information of a plurality of users;
notifying, by the processor, each of the selected plurality of comparison target users of a comparison counterpart user;
acquiring, by the processor, an index value correlated to a state of health of each of the selected plurality of comparison target users; and
setting, by the processor, the acquired index value to a state of being sharable between the plurality of comparison target users, wherein
in the selecting the plurality of comparison target users, a plurality of users scheduled to perform a common action during a predetermined time segment is selected as the comparison target users from the schedule information.

8. A non-transitory storage medium storing a program to cause a processor included in the healthy behavior support device according to claim 1 to execute the individual processing.

9. A healthy behavior support method executed by a device including at least one processor, comprising:
selecting, by the processor, a plurality of comparison target users satisfying a predetermined condition from schedule information of a plurality of users;
notifying, by the processor, each of the selected plurality of comparison target users of a comparison counterpart user;
acquiring, by the processor, an index value correlated to a state of health of each of the selected plurality of comparison target users; and
setting, by the processor, the acquired index value to a state of being sharable between the plurality of comparison target users, wherein
in the selecting the plurality of comparison target users, a plurality of users not having performed a common action longer than a predetermined period of time is selected as the comparison target users from the schedule information.
